(19) United States
(12) Patent Application Publication
(10) Pub. No.: US 2023/0151326 A1
(43) Pub. Date: May 18, 2023

(54) Simplified Compositions and Methods for Generating Neural Stem Cells from Human Pluripotent Stem Cells

(71) Applicant: Wisconsin Alumni Research Foundation, Madison, WI (US)

(72) Inventors: Randolph S. Ashton, Madison, WI (US); Ethan S. Lippmann, Madison, WI (US)

(21) Appl. No.: 17/973,020

(22) Filed: Oct. 25, 2022

Related U.S. Application Data
(62) Division of application No. 15/942,788, filed on Apr. 2, 2018, now abandoned.
Application No. 15/942,788 is a continuation of application No. 13/795,485, filed on Mar. 12, 2013, now abandoned.
(60) Provisional application No. 61/726,382, filed on Nov. 14, 2012.

(51) Int. Cl. C12N 5/0793

(52) U.S. Cl. CPC C12N 5/0619; C12N 2500/90; C12N 2501/15; C12N 2501/155; C12N 2501/727; C12N 2506/02; C12N 2506/45; C12N 2533/52

(57) ABSTRACT

Simplified methods and compositions for directed differentiation of human pluripotent stem cells into neural stem cells are described. Methods and compositions for deriving neural stem cells from human pluripotent stem cells under defined, xeno-free conditions are also described.

### CROSS-REFERENCE TO RELATED APPLICATIONS

This application is a continuation of U.S. application Ser. No. 13/795,485, filed Mar. 12, 2013, which claims priority to U.S. Provisional Patent Application No. 61/726,382 filed on Nov. 14, 2012, each of which is incorporated by reference herein in its entirety.

### BACKGROUND

Human pluripotent stem cells (hPSCs), including human embryonic stem cells (hESCs) and induced pluripotent stem cells (hiPSCs), are an incredibly powerful tool for studying human development and disease and may one day serve as a cell source for regenerative medicine. Significant advancements have been made in the generation of neural stem cells from hPSCs and differentiation to diverse neural lineages of the central nervous system (CNS) and peripheral nervous system (PNS). However, most hPSC neural differentiation protocols to date utilize undefined or undesired or expensive culture components for cell maintenance and differentiation, such as fibroblast feeder layers, undefined extracellular matrix protein coatings (e.g., Matrigel®), or knockout serum replacement. Many of these protocols require manual enrichment steps to purify the resultant neural stem cells, which is undesirable for scale-up. Further, even recent protocols which perform differentiation under chemically defined conditions still utilize hPSCs maintained on MEFs, which limits their clinical utility. Therefore, adaptation of neural differentiation protocols to xeno-free systems is a desirable step for the translation of hPSCs to regenerative therapy and could assist in standardizing differentiation procedures from lab-to-lab by limiting exposure of hPSCs to unknown, animal-derived factors in the undifferentiated state. Thus, there is an ongoing need for completely defined, xeno-free systems for generating neuroepitithelium/neural stem cells from human pluripotent stem cells.

### BRIEF SUMMARY OF THE INVENTION

The invention relates generally to compositions, systems, and methods for generating neural stem cells from human pluripotent stem cells.

Accordingly, in a first aspect disclosed herein is a serum-free medium that supports differentiation of human pluripotent stem cells into neural stem cells (neural differentiation medium), the serum-free medium comprising water, salts, amino acids, vitamins, a carbon source, a buffering agent, selenium, and insulin, wherein the serum-free medium is substantially free of: a TGFβ superfamily agonist, an albumin, and at least one of putrescine and progesterone.

In some embodiments, the serum-free medium further includes one or more of ascorbate, transferrin, or a retinoid.

In some embodiments, the serum-free medium is substantially free of one or more of a fibroblast growth factor, a TGFβ pathway antagonist, and a BMP pathway antagonist.

In some embodiments, a composition is provided that includes human neural stem cells generated and any of the above-described serum-free media that support differentiation of human pluripotent stem cells into neural stem cells. In some embodiments the neural stem cells in the composition are adhering to a substrate. In other embodiments the neural stem cells are in suspension.

In a second aspect described herein is a serum-free medium that supports differentiation of human pluripotent stem cells into neural stem cells (neural differentiation medium), the medium consisting essentially of water, salts, amino acids, vitamins, a carbon source, a buffering agent, selenium and insulin.

In a third aspect disclosed herein is a concentrated supplement for generating a serum-free medium that supports differentiation of human pluripotent stem cells into neural stem cells, the concentrated supplement comprising the ingredients selenium and insulin, wherein the concentration of the ingredients is at least about five fold higher to about 100 fold higher than in the serum-free medium that supports differentiation of pluripotent stem cells into neural stem cells; and wherein the concentrated supplement is substantially free of a TGFβ superfamily agonist, an albumin, and at least one of putrescine and progesterone.

In some embodiments, the concentrated supplement further includes one or more of ascorbate, a transferrin, and a retinoid.

In some embodiments, a kit is provided that includes the concentrated supplement and instructions on a method to differentiate pluripotent stem cells cultured in a monolayer into neural stem cells with the serum-free medium, wherein the serum-free medium is substantially free of a TGFβ pathway antagonist or BMP pathway antagonist.

In a fourth aspect described herein is a system for directed differentiation of human pluripotent stem cells into neural stem cells, the system comprising (i) a solid support comprising a substrate suitable for growth and maintenance of pluripotent stem cells; and (ii) a serum-free medium comprising water, salts, amino acids, vitamins, a carbon source, a buffering agent, selenium and insulin, wherein the serum-free medium is substantially free of: a TGFβ superfamily agonist, an albumin, and at least one of putrescine and progesterone.

In some embodiments the serum-free medium in the system for directed differentiation also includes ascorbate. In some embodiments the solid support includes beads (e.g., microcarriers).

In a fifth aspect disclosed herein is a method for directed differentiation of human pluripotent stem cells into neural stem cells, comprising culturing pluripotent stem cells on a substrate that supports proliferation of pluripotent stem cells, and in a serum-free medium comprising water, salts, amino acids, vitamins, a carbon source, a buffering agent, selenium and insulin, wherein the serum-free medium is substantially free of: a TGFβ superfamily agonist, an albumin, and at least one of putrescine and progesterone.

In some embodiments the substrate to be used is a xenogen-free (xeno-free) substrate. In one embodiment the xeno-free substrate comprises vitronectin, a vitronectin fragment, a vitronectin peptide. In another embodiment the xeno-free substrate comprises a self-coating material. In one embodiment the self-coating material is Synthemax®. In other embodiments, the substrate comprises Matrigel®.

In some embodiments the method also includes a step of passaging the human pluripotent stem cells at least once in the absence of a feeder layer prior to the step of culturing in the serum-free medium.

In some embodiments the human pluripotent stem cells to be used in the method were previously passaged at least once in the absence of a feeder layer prior to culturing in the serum-free medium.

In some embodiments the human pluripotent stem cells are cultured in the substantial absence of a TGFβ signaling antagonist or a BMP antagonist.

In some embodiments, the human pluripotent stem cells are cultured in the serum-free medium for at least 4 to about 6 days. In some embodiments at least 90% of the cultured cells are PAX6-positive at any period during the differentiation from about four days to about six days after beginning the culture step.

In a sixth aspect disclosed herein is a method for directed differentiation of human pluripotent stem cells into neural stem cells, comprising culturing pluripotent stem cells on a substrate that supports proliferation of pluripotent stem cells, and in a serum-free medium consisting essentially of water, salts, amino acids, vitamins, a carbon source, a buffering agent, selenium and insulin.

In a seventh aspect disclosed herein is a method for directed differentiation of human pluripotent stem cells into neural stem cells, comprising culturing pluripotent stem cells on a substrate that supports proliferation of pluripotent stem cells, and in a serum-free medium comprising water, salts, amino acids, vitamins, a carbon source, a buffering agent, selenium and insulin, wherein human pluripotent stem cells are cultured in the substantial absence of any of the following: embryoid bodies, a TGFβ superfamily agonist, a TGFβ signaling antagonist, or a BMP signaling antagonist.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIGS. 1A-1C (a) shows immunofluorescence images of H9 hESCs immunostained for pluripotency markers SOX2 and OCT4; (b) immunofluorescence images of H9 hESCs immunostained for SOX2 and NANOG (Scale bar=50 μm); (c) Flow cytometry histograms for NANOG and OCT 4 (overlapping) at greater than 98% purity; with IgG control on the left.

FIGS. 2A-2D Neuroectoderm differentiation from hPSCs. (a) Schematic overview and timeline of an exemplary embodiment of the disclosed neural differentiation methods. (b) RT-PCR time course analysis of pluripotency, mesoderm, endoderm, and neuroectoderm gene expression starting in undifferentiated H9 hESCs and various time points differentiation into neuroectoderm. (c) Flow cytometry analysis of PAX6. Data is presented as mean±S.D. calculated from at least two biological replicates. Differentiation was conducted on Matrigel® unless otherwise specified. “SB” indicates addition of SB431542 and “N” indicates addition of noggin. (d) Observations of neural rosette formation from H9 hESCs after 6 days of differentiation. E6 (VTN-NC) indicates cells derived from hESCs maintained and differentiated on recombinant vitronectin peptide, a vitronectin fragment, or vitronectin peptide (scale bar, 50 μm). All other images indicate progeny of hESCs maintained and differentiated on Matrigel® (scale bars, 250 μm).

FIG. 3 shows line charts depicting time course of NANOG expression changes, evaluated by flow cytometry, during neural differentiation (days 1-6) in E6 medium of various hPSC lines and cell culture substrates. Top curve (triangle symbols) depicts data for H9 hESCs cultured on vitronectin (VTN-NC); second curve (circles) depicts data for H1 hESCs cultured on VTN-NC; third curve (diamonds) depicts data for H9 hESCs cultured on Matrigel®; and the fourth curve (squares) depicts data for IMR90-4 hiPSCs cultured on Matrigel®. Data represent mean±S.D. from two biological replicates.

FIGS. 4A-4B (a) shows flow cytometry histograms demonstrating uniform labeling of N-Cadherin, OTX2, and SOX2. Mean±S.D. are provided in the Examples section herein and are representative of two biological replicates. Histograms on the right show data for protein of interest; histograms on the left show IgG control data. (b) Immunofluorescence images for neural stem cell markers, OTX2 and SOX2 in neural stem cells obtained from hPSCs generated by the disclosed methods. Adjacent panels represent the same field. Scale bars, 50 μm.

FIGS. 5A-5B (a) PAX6 and SOX1 expression were examined by immunocytochemistry at various differentiation time points in E6 medium. RA=retinoic acid. Scale bars, 100 μm. (b) SOX1 expression was examined by flow cytometry at 9 days of differentiation in E6 medium or E6 medium containing 1 μM RA. Representative histograms are provided, whereas mean±S.D. were calculated from two biological replicates. Right-shifted histogram, Sox1; Left-shifted histogram, IgG control.

FIG. 6 shows an immunofluorescence image (PAX6 and N-Cadherin) of neural rosettes obtained by differentiating H1 hESCs for 6 days in E6 medium. Scale bar, 250 μm.

FIG. 7 Comparison of neuroectoderm formation in H9 hESCs maintained and differentiated under various conditions. H9 hESCs maintained on Matrigel® or VTN-NC in E8 medium were differentiated in E6 medium under adherent conditions or free-floating EBs as described in the Examples section. Alternatively, hESCs were maintained on MEFs in unconditioned medium and differentiated in E6 medium under adherent conditions. After 6 days of differentiation, all cultures exhibited regions with columnar or polarized neuroepithelial morphology (scale bars, 50 μm). Cultures were also probed for PAX6 expression at this time point (filled histogram, IgG control; unfilled histogram, PAX6⁺ cells). Percentages were calculated from two biological replicates.

FIG. 8 Effect of hPSC seeding density on differentiation. Cells were seeded onto Matrigel® or vitronectin (VTN-NC) at the indicated densities and differentiated for 6 days. Cells seeded on VTN-NC at 1×10⁴ cells/cm² were not analyzed by flow cytometry due to limited outgrowth. Mean±S.D. was calculated from two biological replicates.

FIG. 9 Flow cytometry for PAX6 in neural progenitors obtained by differentiation of H9 hESCs plated on Matrigel® and cultured for 6 days in E6 medium; E5 medium (no transferrin); or E5 medium (no insulin).

FIG. 10 Flow cytometry for OTX2 in neural progenitors obtained by differentiation of H9 hESCs plated on Matrigel® and cultured for 6 days in E6 medium; E5 medium (no transferrin); or E5 medium (no insulin).

FIG. 11 Flow cytometry for N-Cadherin in neural progenitors obtained by differentiation of H9 hESCs plated on Matrigel® and cultured for 6 days in E6 medium; E5 medium (no transferrin); or E5 medium (no insulin).

FIG. 12 Flow cytometry for SOX2 in neural progenitors obtained by differentiation of H9 hESCs plated on Matrigel® and cultured for 6 days in E6 medium; E5 medium (no transferrin); or E5 medium (no insulin).

FIG. 13 Bright field image of neural progenitors obtained after differentiation of H9 hESCs for 6 days in E6 medium, E5 medium (no transferrin); or E5 medium (no insulin).

FIG. 14 Flow cytometry for PAX6 in neural progenitors obtained by differentiation of H9 hESCs plated at 1×10⁵ cells/cm in E8 medium plus ROCK inhibitor on vitronectin (VTN-NC) and cultured starting the day after plating for 6 days in E4 medium that contained DMEM/F12, bicarbonate, ascorbate, and insulin, but was substantially free of selenium.

FIG. 15 Flow cytometry for PAX6, N-cadherin, and SOX2 (left to right) in neural progenitors obtained by differentiation of H9 hESCs plated at 1×10⁵ cells/cm in E8 medium plus ROCK inhibitor on vitronectin (VTN-NC), and cultured starting the day after plating for 6 days in E4 medium that contained DMEM/F12, bicarbonate, selenium, and insulin, but was substantially free of ascorbate.

FIG. 16 Flow cytometry for PAX6 in neural progenitors obtained by differentiation of H9 hESCs plated at 1×10⁵ cells/cm in E8 medium plus ROCK inhibitor on vitronectin (VTN-NC) and cultured starting the day after plating for 6 days in E4 medium containing DMEM/F12, bicarbonate, selenium, and an insulin concentration of approximately 5 mg/L, a four fold lower concentration of insulin than present in E6 medium.

FIG. 17 Bright field image of neural progenitors obtained after differentiation of H9 hESCs for 6 days in E4 medium that contained no selenium. Neural progenitors exhibit a loss of viability in the absence of selenium.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

The present invention relates to the inventors' unexpected finding that a minimal set of cell culture conditions and components, e.g., xeno-free cell culture components, can be used to generate neuroepithelium/neural stem cells from human pluripotent stem cells, in the absence of TGFβ pathway antagonists, BMP pathway antagonists, or embryoid bodies. Among the advantages of the described methods and compositions is the ability to generate human neural stem cells under completely defined and xeno-free conditions with a minimal number of components. The reduced number of components needed for neural differentiation reduces costs and increases the consistency of neural differentiation from human pluripotent stem cells.

### I. Definitions

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

In describing the embodiments and claiming the invention, the following terminology will be used in accordance with the definitions set out below.

As used herein, the term human “pluripotent stem cell” (hPSC) means a cell capable of continued self-renewal and of capable, under appropriate conditions, of differentiating into cells of all three germ layers. Examples of hPSCs include human embryonic stem cells (hESCs) and human induced pluripotent stem cells (hiPSCs). As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g. multipotent or somatic) cells.

As used herein, “differentiation efficiency” refers to the proportion of cells in a population that are PAX6⁺ neural stem cells.

As used herein, “xeno-free” refers to xenogen-free, meaning in the substantial absence of undefined components that are derived from a non-human source.

As used herein, “neural stem cell” refers to a multipotent stem cell that is PAX6⁺ and is capable of differentiating into neurons or astrocytes of the CNS or PNS.

As used herein, “about” means within 5% of a stated concentration range or within 5% of a stated time frame.

As used herein, “serum-free” means that a medium does not contain serum or serum replacement, or that it contains essentially no serum or serum replacement. For example, an essentially serum-free medium can contain less than about 1%, 0.9%, 0.8%, 0.7%, 0.6%, 0.5%, 0.4%, 0.3%, 0.2% or 0.1% serum, wherein the culturing capacity of the medium is still observed.

As used herein, “substantially free of putrescine” means no putrescine is added to a cell culture medium above and beyond any putrescine present in the base medium, e.g., DMEM/F12. Alternatively, “substantially free of putrescine” means a final putrescine concentration less than or equal to 0.08 mg/L.

The term “defined culture medium” or “defined medium,” as used herein, means that the chemical structure and quantity of each medium ingredient is definitively known.

As used herein, “a medium consisting essentially of” means a medium that contains the specified ingredients and those that do not materially affect its basic characteristics.

As used herein, “effective amount” means an amount of an agent sufficient to evoke a specified cellular effect according to the present invention.

As used herein, “viability” means the state of being viable. Pluripotent cells that are viable attach to the cell plate surface and do not stain with the dye propidium iodide absent membrane disruption. Short term viability relates to the first 24 hours after plating the cells in culture. Typically, the cells do not proliferate in that time.

As used herein, “pluripotency” means a cell's ability to differentiate into cells of all three germ layers.

### II. Compositions

Cell Culture Media for Neural Differentiation of hPSCs into Neural Stem Cells

Described herein are new simplified media specifically formulated to support differentiation of hPSCs into neural stem cells. Various media components, such as salts, vitamins, glucose sources, minerals, and amino acids were tested, alone or in combination, to determine simpler neural differentiation media formulations than those previously described in the art.

The media described herein are able to support differentiation of hPSCs into neural stem cells, as assessed by the expression pattern of a number of cell type-associated markers as described herein. A list of components for exemplary media described herein is set forth in Table 1.

TABLE 1
Exemplary Simplified Neural Differentiation Media Components

| Formulation | 1 | 2 | 3 | 4 |
|---|---|---|---|---|
| DMEM/F12* | + | + | + | + |
| Selenium | + | + | + | + |
| Insulin | + | + | + | + |
| L-Ascorbic Acid | − | + | − | + |
| (Ascorbate) | | | | |
| Transferrin | − | − | + | + |

*or similar basal medium buffered to physiological pH (about 7.4) with bicarbonate or another suitable buffer such as HEPES. Osmolarity of the medium was adjusted to about 340 mOsm.

The final concentrations of the above listed components in the above listed exemplary media are listed in Table 2:

TABLE 2
Concentrations of Components Found in Exemplary
Neural Differentiation Media Compositions

| | Final | |
|---|---|---|
| Component | Concentration | |
| Sodium Selenite | 14 | μg/L |
| Insulin | 19.4 | mg/L |
| L-Ascorbic Acid | 64 | mg/L |
| Transferrin | 10.7 | mg/L |

The various neural differentiation media described herein can be prepared from the basic ingredients. Alternatively, one of skill in the art appreciates the efficiency of using a basal medium such as DMEM/F12 as starting material to prepare the disclosed neural differentiation media. The term “basal medium” as used herein means a medium that supports the viability and growth of cells that do not require special media additives. Typical basal medium components are known in the art and include salts, amino acids, vitamins, a carbon source (e.g., glucose), and a buffer. Other components that do not change the basic characteristic of the medium but are otherwise desirable can also be included, such as the pH indicator phenol red. For example, Dulbecco's Modified Eagle Medium: Nutrient Mixture F-12 (DMEM/F12) is a basal medium commonly used to make suitable growth media for mammalian cell culture. A complete list of ingredients of DMEM/F12 is set forth in Table 3.

TABLE 3
DMEM: F-12 Medium Formulation (ATCC Catalog No. 30-2006).

| Inorganic Salts (g/liter) | Amino Acids (g/liter) | Vitamins (g/liter) | Other (g/liter) |
|---|---|---|---|
| CaCl2 (anhydrous) | L-Alanine 0.00445 | D-Biotin 0.00000365 | D-Glucose 3.15100 |
| 0.11665 | L-Arginine•HCl 0.14750 | Choline Chloride 0.00898 | HEPES 3.57480 |
| CuSO4 (anhydrous) | L-Asparagine•H2O | Folic Acid 0.00265 | Hypoxanthine 0.00239 |
| 0.0000008 | 0.00750 | myo-Inositol 0.01261 | Linoleic Acid 0.000044 |
| Fe(NO3)3•9H2O 0.00005 | L-Aspartic Acid 0.00665 | Niacinamide 0.00202 | Phenol Red, Sodium Salt |
| FeSO4•7H2O 0.000417 | L-Cystine•HCl•H2O | D-Pantothenic Acid | 0.00810 |
| MgSO4 (anhydrous) | 0.01756 | 0.00224 | Putrescine•2HCl 0.00008 |
| 0.08495 | L-Cystine•2HCl 0.03129 | Pyridoxine•HCl 0.00203 | Pyruvic Acid•Na 0.05500 |
| KCl 0.3118 | L-Glutamic Acid 0.00735 | Riboflavin 0.00022 | DL-Thioctic Acid |
| NaHCO3 1.20000 | L-Glutamine 0.36510 | Thiamine•HCl 0.00217 | 0.000105 |
| NaCl 7.00000 | Glycine 0.01875 | Vitamin B-12 0.00068 | Thymidine 0.000365 |
| Na2HPO4 (anhydrous) | L-Histidine•HCl•H2O | | |
| 0.07100 | 0.03148 | | |
| NaH2PO4•H2O 0.06250 | L-Isoleucine 0.05437 | | |
| ZnSO4•7H2O 0.000432 | L-Leucine 0.05895 | | |
| | L-Lysine•HCl 0.09135 | | |
| | L-Methionine 0.01724 | | |
| | L-Phenylalanine 0.03548 | | |
| | L-Proline 0.01725 | | |
| | L-Serine 0.02625 | | |
| | L-Threonine 0.05355 | | |
| | L-Tryptophan 0.00902 | | |
| | L-Ty rosine · 2Na · 2H2O | | |
| | 0.05582 | | |
| | L-Valine 0.05285 | | |

In some embodiments a serum-free medium that supports differentiation of human pluripotent stem cells into neural stem cells (“neural differentiation medium”) includes water, salts, amino acids, vitamins, a carbon source, a buffering agent, selenium, and insulin, but the medium (referred to as an “E4” medium) is substantially free of a TGFβ superfamily agonist (e.g., Nodal), an albumin, and at least one of putrescine and progesterone.

In some embodiments the concentration of selenium ranges from about 2 μg/L to about 80 μg/L, e.g., 4 μg/L, 6 μg/L, 8 μg/L, 10 μg/L, 12 μg/L, 15 μg/L 20 μg/L, 25 μg/L, 30 μg/L, 40 μg/L, 50 μg/L, 60 μg/L, 75 μg/L or another concentration of selenium from about 2 μg/L to about 80 μg/L. In one embodiment, the concentration of selenium is 14 μg/L.

In some embodiments the concentration of insulin used in the neural differentiation medium ranges from about 1 mg/L to about 50 mg/L, e.g., 2 mg/L, 3 mg/L, 5 mg/L, 7 mg/L, 8 mg/L 10 mg/L, 15 mg/L, 20 mg/L, 25 mg/L, 35 mg/L, 40 mg/L, or another concentration of insulin from about 1 mg/L to about 50 mg/L. In one embodiment, the concentration of insulin is 19.4 mg/L.

In other embodiments, the neural differentiation medium includes water, salts, amino acids, vitamins, a carbon source, a buffering agent, selenium, insulin, and ascorbic acid (ascorbate), but the medium is substantially free of a TGFβ superfamily agonist (e.g., Nodal), an albumin, and at least one of putrescine and progesterone.

In some embodiments, the concentration of ascorbate used in the medium ranges from about 10 mg/L to about 200 mg/L, e.g., 15 mg/L, 25 mg/L, 30 mg/L, 40 mg/L, 50 mg/L, 60 mg/L, 75 mg/L, 80 mg/L, 100 mg/L, 125 mg/L, 150 mg/L, 175 mg/L, or another concentration of ascorbate from about 10 mg/L to about 200 mg/L. In one embodiment, the concentration of ascorbate is 64 mg/L. As is known in the art, cell culture media should be buffered to a physiological pH of about 7.4. A number of agents suitable as pH buffers include, but are not limited to, bicarbonate, HEPES, TAPSO, or another Good's buffer suitable for buffering to a physiological pH of about 7.2 to about 7.6.

In some cases, the neural differentiation medium includes additional components. Exemplary, non-limiting concentrations of some of these components are listed in Table 2.

In some embodiments, ascorbate is also included. In other embodiments, transferrin is also included. In some embodiments, transferrin can range in concentration from about 2 mg/L to about 50 mg/L, e.g., about 3 mg/L, 7 mg/L, 8 mg/L, 10 mg/L, 11 mg/L, 12 mg/L, 15 mg/L, 20 mg/L, 25 mg/L, 30 mg/L, 35 mg/L, 40 mg/L, or another concentration of transferrin from about 2 mg/L to about 50 mg/L. In one embodiment, the concentration of transferrin is 10.7 mg/L.

In some embodiments both ascorbate and transferrin are included. In one embodiment, where both ascorbate and transferrin are included, bicarbonate is used as the buffer, and the medium is referred to as “E6” medium as described herein. In another embodiment, where the medium has the same composition as that of E6, but excludes transferrin, the medium is referred to as an “E5” medium. In some embodiments, where the medium comprises, at a minimum, a buffering agent (e.g., bicarbonate), base medium (e.g., DMEM/F12), insulin, and selenium, the medium is referred to as an “E4” medium.

In some embodiments, the medium comprises a buffering agent (e.g., bicarbonate), base medium (e.g., DMEM/F12), insulin (19.4 mg/L), and selenium (14 μg/L).

In other embodiments, the medium comprises a buffering agent (e.g., bicarbonate), base medium (e.g., DMEM/F12), insulin (19.4 mg/L), selenium (14 μg/L), and L-Ascorbic acid (64 mg/L).

In other embodiments, the medium comprises a buffering agent (e.g., bicarbonate), base medium (e.g., DMEM/F12), insulin (19.4 mg/L), selenium (14 μg/L), L-Ascorbic acid (64 mg/L), and transferrin (10.7 mg/L).

In some embodiments, the concentration of components in the medium will be as indicated in Table 2, except for one component, the concentration of which will fall within a range as described herein. In other embodiments, the concentration of more than one of the components can vary from that indicated in Table 2, but will fall within concentration ranges as described herein.

In some embodiments, the neural differentiation medium is also substantially free of certain other components. In some embodiments, a fibroblast growth factor (FGF), e.g., FGF2 is substantially excluded. In other embodiments, neural differentiation medium is substantially free of a TGFβ pathway antagonist or BMP pathway antagonist.

Optionally, a fibroblast growth factor (e.g., FGF2) may also be included in the medium to be used (e.g., at an exemplary concentration of about 20 ng/ml). In some embodiments, a retinoid (e.g., all-trans retinoic acid) is also included to facilitate neural differentiation into certain neuronal lineages depending on the concentration of retinoid used. In some embodiments, the concentration of the retinoid, e.g., all-trans retinoic acid, is about 0.1 μM to about 1.0 μM.

In other embodiments, the serum-free also includes a tumor growth factor β (TGFβ) signaling antagonist (e.g., SB431542, Sigma; at about 5-15 μM, e.g., 10 μM) and a bone morphogenetic protein (BMP) signaling antagonist (e.g., noggin at about 200 ng/ml or dorsomorphin at about 1 μM).

In some embodiments, the neural differentiation medium is a serum-free medium that consists essentially of water, salts, amino acids, vitamins, a carbon source, a buffering agent, selenium, and insulin, and is referred to herein as “E4” medium. In some embodiments, the neural differentiation medium optionally includes ascorbate.

### Cell-Based Compositions

In some embodiments, upon differentiation of hPSCs into neural stem cells using the neural differentiation media described herein, a cellular composition is obtained comprising human neural stem cells and any of the neural differentiation media disclosed herein. In some embodiments, the neural stem cells in the composition are adhering to a substrate, e.g., a xeno-free substrate such as vitronectin. In other embodiments, the neural stem cells in the composition are in suspension.

### Concentrated Supplements

In some embodiments described herein is a concentrated supplement for generating a serum-free medium that supports differentiation of human pluripotent stem cells into neural stem cells, the concentrated supplement comprising the ingredients selenium and insulin, wherein the concentration of the ingredients is at least about five fold higher to about 100 fold higher than in the serum-free medium that supports differentiation of pluripotent stem cells into neural stem cells; and wherein the concentrated supplement is substantially free of a TGFβ superfamily agonist, an albumin, and at least one of putrescine and progesterone.

Optionally, the concentrated supplement can include ascorbate, transferrin, or both. In one embodiment, the concentrated supplement also includes a retinoid (e.g., retinol acetate or all-trans retinoic acid).

A neural differentiation medium can be obtained by diluting the concentrated supplement in a base medium, e.g., DMEM-F12 with a suitable dilution, depending on the initial concentration of the concentrated supplement. The pH of the neural differentiation medium so obtained is then adjusted to about pH 7.4 with addition of a suitable buffer, e.g., bicarbonate or HEPES, plus acid or base.

The concentration of components in the concentrated supplement may range from about five fold higher to about 200 fold higher than their final concentration in the neural differentiation medium, e.g., about 6, 10, 20, 30, 40, 50, 70, 80, 100, 120, 150, 180, or another fold higher than their final concentration in the neural differentiation medium obtained by dilution of the concentrated supplement in a basal medium. In one embodiment, the components in the concentrated supplement are at a 100 fold higher concentration than their final concentration after dilution in base medium, i.e., the concentrated supplement is a “100×” supplement. In another embodiment, the concentrated supplement is a 50×supplement. In another embodiment, the concentrated supplement is a 200× supplement.

In some embodiments, any of the above-described concentrated supplements is provided as part of a kit, where the kit includes the concentrated supplement itself plus instructions on a method, as described herein, to differentiate pluripotent stem cells cultured in a monolayer into neural stem cells with the serum-free medium, wherein the serum-free medium is obtained by dilution of the concentrated supplement in a base medium and is substantially free of a TGFβ pathway antagonist or BMP pathway antagonist. In some embodiments, one or more of the components of the concentrated supplement are provided separately within the kit. For example, in some cases, insulin, transferrin, or a retinoid are provided separately from the remaining components and are added in separately at an appropriate dilution to a neural differentiation medium upon dilution of the concentrated supplement.

### Systems

Also disclosed herein is a system for directed differentiation of human pluripotent stem cells into neural stem cells, the system comprising (i) a solid support comprising a substrate suitable for growth and maintenance of pluripotent stem cells; and any of the neural differentiation media disclosed herein. Suitable solid supports include any cell culture vessels (e.g., dishes, flasks, multiwell plates, and the like) and microcarrier beads coated with a suitable substrate, e.g., GEM™ microcarrier beads (Hamilton), which are useful for large scale growth and neural differentiation of hPSCs in suspension in a bioreactor. Suitable substrates include, e.g., Matrigel®, vitronectin, a vitronectin fragment, or a vitronectin peptide, a Synthemax® substrate (or another type of self-coating substrate).

### III. Methods

In various embodiments, hPSCs, e.g., hESCs or hiPSCs, are cultured in the absence of a feeder layer (e.g., a fibroblast layer) on a substrate suitable for proliferation of hPSCs, e.g., Matrigel®, vitronectin, a vitronectin fragment, or a vitronectin peptide, or Synthemax®, prior to plating for neural differentiation. In some cases, the hPSCs are passaged at least 1 time to at least about 5 times in the absence of a feeder layer. Suitable culture media for passaging and maintenance of hPSCs include, but are not limited to, mTeSR® and E8™ media. In some embodiments, the hPSCs are maintained and passaged under xeno-free conditions, where the cell culture medium is a defined medium such as E8 or mTeSR, but the cells are maintained on a completely defined, xeno-free substrate such as vitronectin, or Synthemax® (or another type-of self-coating substrate).

In one embodiment, the hPSCs are maintained and passaged in E8 medium on vitronectin, a vitronectin fragment, or a vitronectin peptide or a self-coating substrate such as Synthemax®.

Typically, to increase plating efficiency and cell viability hPSCs are initially plated one of the above-mentioned feeder-free substrates in one of the above-mentioned media in the presence of a Rho-Kinase (ROCK) inhibitor, e.g., Y-27632 (R&D Systems) at a concentration of about 10 μM and cultured overnight prior to initiating neural differentiation.

In preparation for neural differentiation as described herein, hPSCs are typically plated at a density of at least about 1×10⁵ cells/cm² to about 2×10⁵ cells/cm², whereby the cells will be at least about 95% confluent upon changing the medium from one suited for hPSC proliferation to one that sustains differentiation of the hPSCs as described herein. While not wishing to be bound by theory, it is believed that the density of hPSCs is an important factor affecting the efficiency of the methods described herein.

In various embodiments, the differentiation of hPSCs into neural stem cells is effected by culturing the PSCs in any of a number of serum-free media that support differentiation of human pluripotent stem cells into neural stem cells, collectively referred to herein as (“neural differentiation media”).

In some embodiments, the neural differentiation medium to be used in the neural differentiation method is “E4” medium, which consists essentially of a base medium (e.g., DMEM/F12 or a similar base medium as described herein) containing water, salts, amino acids, vitamins, a carbon source, a buffering agent; plus selenium and insulin. Optionally, the neural differentiation medium to be used may also include ascorbate (referred to herein as an “E5” medium).

In other embodiments the medium to be used includes at least the same components as the E4 medium mentioned above, but the medium is substantially free of: a TGFβ superfamily agonist (e.g., Nodal); an albumin, and at least one of putrescine and progesterone. In some embodiments the medium to be used is an E4 medium plus ascorbate. In other embodiments, the medium to be used is an E4 medium plus transferrin. In some embodiments, the medium to be used is E4 medium plus ascorbate and transferrin. In one embodiment the differentiation medium to be used is a carbonate-buffered E4 medium plus ascorbate and transferrin, which is also referred to herein as an “E6” medium. Optionally, a fibroblast growth factor (e.g., FGF2) may also be included in the medium to be used. In other embodiments, the medium to be used does not include a fibroblast growth factor. In some embodiments, a retinoid (e.g., all-trans retinoic acid) is also included to facilitate neural differentiation into certain neuronal lineages depending on the concentration of retinoid used. In some embodiments, the concentration of the retinoid, e.g., all-trans retinoic acid, is about 0.1 μM to about 1.0 μM.

In some embodiments, the medium to be used does not include a transforming growth factor β (TGFβ) signaling antagonist or a bone morphogenetic protein (BMP) signaling antagonist. In other embodiments, the medium to be used is an E4 medium in combination with a transforming growth factor β (TGFβ) signaling antagonist (e.g., SB431542, Sigma; at about 10 μM) and a bone morphogenetic protein (BMP) signaling antagonist (e.g., noggin at about 200 ng/ml or dorsomorphin at about about 1 μM).

In other embodiments, directed differentiation of human pluripotent stem cells into neural stem cells, is carried out by culturing pluripotent stem cells on a substrate that supports proliferation of pluripotent stem cells (e.g., vitronectin or Matrigel®), and in a serum-free medium comprising water, salts, amino acids, vitamins, a carbon source, a buffering agent, selenium and insulin, wherein human pluripotent stem cells are cultured in the substantial absence of embryoid bodies, a TGFβ superfamily agonist, a TGFβ signaling antagonist, or a BMP signaling antagonist.

In the neural differentiation methods described herein, a number of suitable substrates can be used to culture hPSCs in the process of differentiation into neural stem cells. In some embodiments, the substrate to be used is an undefined extracellular matrix protein substrate such as Matrigel®. In other embodiments, a defined, xenogen-free substrate is used. Such substrates include, but are not limited to, vitronectin, a vitronectin fragment, a vitronectin peptide, and self-coating substrates such as Synthemax® (Corning).

In some embodiments, the hPSCs are cultured in the presence of one of the neural differentiation media described herein to obtain a population of cells that is at least about 90% PAX6-positive (by protein expression) within a period of at least about four days to about 12 days, e.g., about 5 days, 6 days, 7 days, 8 days, 9 days, 10 days, 11 days, or another period from at least about 4 days to about 12 days. In some embodiments, the cultured cells are at least 90% PAX6-positive at any period from about four days to about six days after initiating neural differentiation of the hPSCs.

The expression (or lack thereof) of a number of cell type-associated markers can be used to characterize the differentiation of hPSCS into neural stem cells over the course of the methods described herein. For example, the expression of some markers associated with pluripotency in hPSCs decline over the course of differentiation of the hPSCs into neural stem cells. Such pluripotency markers include Oct4, Nanog, SSEA-3, SSEA-4, TRA-1-60, and TRA-1-81. Likewise, certain markers associated with mesoderm or endoderm also decline over time or are absent, e.g., T (Brachyury) and Sox 17. Conversely the RNA expression of markers associated with neural stem cells increases over the course of differentiation. Suitable markers (at the RNA or protein level) for neural stem cells and neural differentiation include, but are not limited to, PAX6, SOX2, Nestin, N-Cadherin, and SOX1.

Suitable methods for evaluating the above-markers are well known in the art and include, e.g., qRT-PCR, RNA-sequencing, and the like for evaluating gene expression at the RNA level. Quantitative methods for evaluating expression of markers at the protein level in cell populations are also known in the art. For example, flow cytometry, is typically used to determine the fraction of cells in a given cell population that express (or do not express) a protein marker of interest (e.g., PAX6).

Typically, the populations of neural stem cells obtained by the methods described herein comprise at least 90% PAX6-positive neural stem cells. Such populations may then be pattered into various cell types within the CNS or PNS. For example, the neural stem cells can be differentiated into motor neurons, forebrain cortical glutamatergic neurons, GABAergic neurons, cholinergic neurons, and astrocytes. Alternatively, the neural stem cells may be passaged and expanded in the presence of FGF and/or frozen in a freezing medium (e.g., Synth-A-Freeze® medium at high density (e.g., about 1×10⁶ cells/ml)

The invention will be more fully understood upon consideration of the following non-limiting Examples.

### EXAMPLES

### Example 1 Materials and Methods

Maintenance of hPSCs

hPSCs were obtained as frozen vials banked under feeder-independent conditions in mTeSR1 medium (STEMCELL Technologies). hPSCs were then thawed and cultured directly into E8 medium consisting of DMEM/F12 (Invitrogen), 64 mg/L ascorbic acid (Sigma), 543 mg/L sodium bicarbonate (Sigma), 14 μg/L sodium selenite (Sigma), 19.4 mg/L insulin (Sigma), 10.7 mg/L transferrin (Sigma), 100 μg/L FGF2 (Waisman Clinical Biomanufacturing Facility, University of Wisconsin-Madison), and 2 μg/L TGFβ1 (Peprotech). hPSCs were maintained on Matrigel® (BD Biosciences) or recombinant vitronectin peptide (VTN-NC) as described in Chen et al (2011), Nature Methods, 8:424-429. Cell lines used in this study were H9 hESCs (passage 25-45), H1 hESCs (passage 28-36), and IMR90-4 iPSCs (passage 26-40). For some comparative experiments, H9 hESCs were maintained on irradiated mouse embryonic fibroblasts (MEFs) in standard unconditioned medium: DMEM/F12 containing 20% Knockout Serum Replacer (Invitrogen), 1×MEM nonessential amino acids (Invitrogen), 1 mM L-glutamine (Sigma), 0.1 mM β-mercaptoethanol (Sigma), and FGF2 (4 ng/mL). Cells were routinely passaged with Versene (Invitrogen) as previously described (Chen et al, supra). The ROCK inhibitor Y-27632 (R&D systems) was included at a final concentration of 1 μM when passaging H1 hESCs onto VTN-NC to facilitate attachment.

### Differentiation to Neuroepithelium Under Adherent Conditions

hPSCs were washed once with phosphate-buffered saline (PBS; Invitrogen), incubated with Accutase® (Invitrogen) for 3 min, and collected by centrifugation. hPSCs were then plated onto Matrigel® or VTN-NC at a density of 2×10⁵ cells/cm² in E8 medium containing 10 μM ROCK inhibitor and cultured overnight. The following morning, cells were changed to E6 medium, E6 containing 10 μM SB431542 (Cellagentech), or E6 containing 10 μM SB431542 and 200 ng/mL recombinant human noggin (R&D Systems) to initiate differentiation. E6 medium is the same formulation as E8 medium but without FGF2 and TGFβ1. Medium was changed every day until cells were utilized for analysis. After these initial experiments, additional seeding densities of 1×10⁵ cells/cm², 5×10⁴ cells/cm², and 1×10⁴ cells/cm² were tested with E6 medium to determine the effect of seeding density on neuroepithelial differentiation.

### Embryoid Body (EB) Formation

To form EBs, hESCs were incubated with 2 mg/mL dispase (Invitrogen) for 10-15 min to facilitate colony detachment, washed twice with DMEM/F12, and transferred to low-attachment 6-well plates (Corning) in E6 medium. The medium was changed every other day. At day 4 of differentiation, whole EBs were transferred to standard tissue culture polystyrene dishes or glass chamber slides coated with Matrigel® Matrigel® or 100 μg/mL poly-L-ornithine (Sigma) and 50 μg/mL laminin (Invitrogen). Resultant cells were maintained in E6 medium for the duration of each experiment.

### Immunocytochemistry

Cells were washed twice with PBS and fixed with 4% paraformaldehyde for 10 min at room temperature. After additional washes in PBS, cells were blocked and permeabilized in TBS-DT (tris-buffered saline (TBS) containing 5% donkey serum (Sigma) and 0.3% Triton X-100 (TX-100; Fisher)) for at least one hour at room temperature. Primary antibodies were diluted in TBS-DT and cells were incubated in these antibodies overnight at 4° C. Antibodies against OCT 3/4 (rabbit; Santa Cruz Biotechnology; 1:100), NANOG (rabbit; Cell Signaling; 1:200), SOX2 (mouse; Millipore; 1:200), Pax6 (rabbit; Covance; 1:1000), SOX1 (goat; R&D Systems; 1:500), N-Cadherin (mouse; BD Biosciences; 1:500), OTX2 (goat; R&D Systems; 1:500), and HOXB4 (rat; DHSB; 1:50) were utilized for immunocytochemistry. The following day, chambers were rinsed once with TBS containing 0.3% Triton X-100 (TBST) and then washed five times, 15 min apiece, with TBST. Donkey anti-mouse Alexa Fluor 488 (Invitrogen; 1:500), donkey anti-goat Alexa Fluor 488 (Invitrogen; 1:500), donkey anti-mouse Cy3 (Jackson ImmunoResearch; 1:500), donkey anti-rat Cy3 (Jackson ImmunoResearch; 1:500), or donkey anti-rabbit Cy3 (Jackson ImmunoResearch; 1:500) were diluted in TBS++ and incubated on the cells for 1 hour at room temperature and nuclei were subsequently counterstained with 300 nM 4′,6-Diamidino-2-pheny-lindoldihydrochloride (DAPI) for 10 min. After three rinses with TBS, cells were washed once for 25 min with TBS and three additional times for 10 min apiece. Cells were then mounted with Prolong Gold Antifade Reagent (Invitrogen) and visualized using a Nikon MR confocal microscope. Nikon NIS-Elements software was used for image analysis.

### Flow Cytometry

Cells were harvested from 6- or 12-well plates by washing once with PBS and incubating with Accutase® for 3-5 min. Cells were then recovered by centrifugation and fixed in 4% paraformaldehyde for 10 min at room temperature. After blocking with PBS containing 10% normal serum (goat or donkey serum depending on the species of primary antibody; Sigma) and 0.1% Triton X-100 for at least 30 min at room temperature, cells were incubated with primary antibodies for 1 hour at room temperature or overnight at 4° C. Antibodies were diluted in PBS containing 10% normal serum and the antibodies used for flow cytometry include PAX6 (mouse; DHSB; 1:1000), NANOG (1:200), SOX2 (1:500), SOX1 (1:500), OTX2 (1:500), N-Cadherin (1:500), and HOXB4 (1:50). IgG controls were included for each species of antibody (Invitrogen). After washing twice with PBS containing 0.75% bovine serum albumin (BSA; Invitrogen), cells were incubated for 30-60 min at room temperature in PBS containing 10% normal serum and 1:200 dilutions of goat anti-rabbit Alexa Fluor 488, goat anti-mouse Alexa Fluor 647, donkey anti-goat Alexa Fluor 488, donkey anti-rat Cy3, or donkey anti-mouse Alexa Fluor 647, depending on the species of primary antibody. After washing twice with PBS containing 0.75% BSA, cells were analyzed on a FACSCanto™ (BD Biosciences) and resultant data were analyzed using Cyflogic software. Positive events were determined by gating the top 1% of the IgG control histograms, and all flow data presented are from biological replicates.

### Reverse Transcriptase Polymerase Chain Reaction (RT-PCR)

Total RNA was extracted from cells using Trizol reagent (Invitrogen) according to the manufacturer's instructions. Five μg of total RNA was then subjected to reverse-transcription using a Thermoscript RT-PCR kit (Invitrogen) in a 20 μL mixture according to the manufacturer's instructions. 0.5 μL of resultant cDNA was then amplified in a 25 μL mixture containing 10×PCR buffer, 0.2 mM dNTP, 1.5 mM MgCl₂, 0.5 μM of each primer, and 1 U Taq DNA polymerase (Invitrogen). Amplified products were resolved on 2% agarose gels containing SYBR® Safe (Invitrogen) and visualized with a VersaDoc™ (Biorad).

### Example 2 hPSCs Maintained in E8 Medium Undergo Rapid Neural Specification in E6 Medium Under Defined Conditions

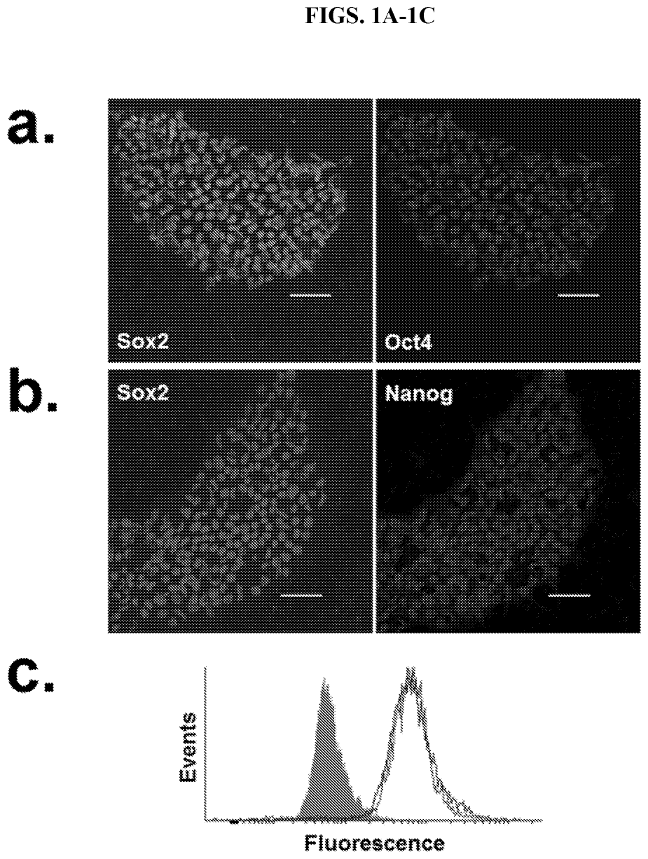

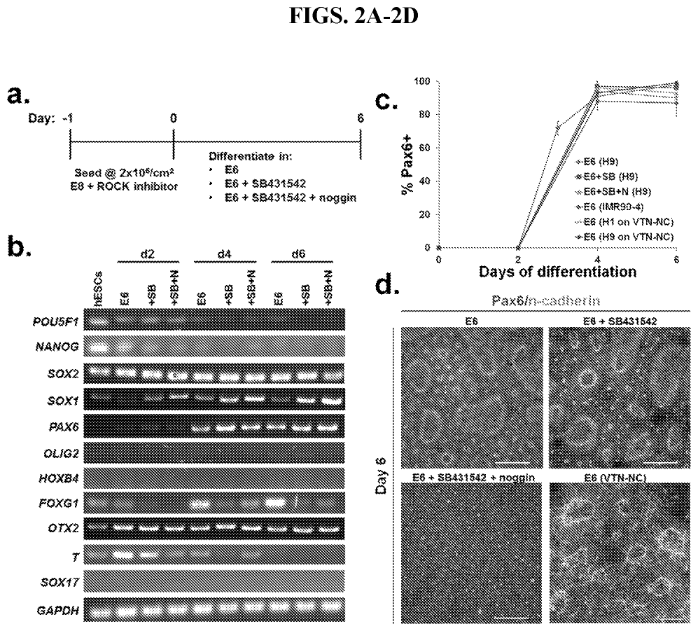

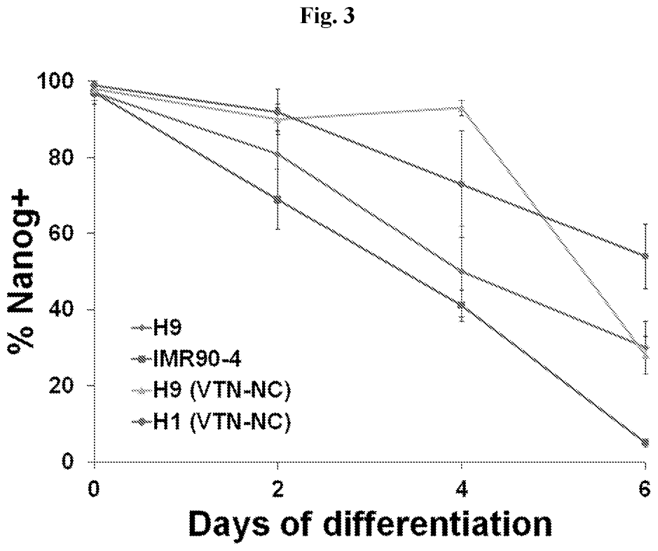

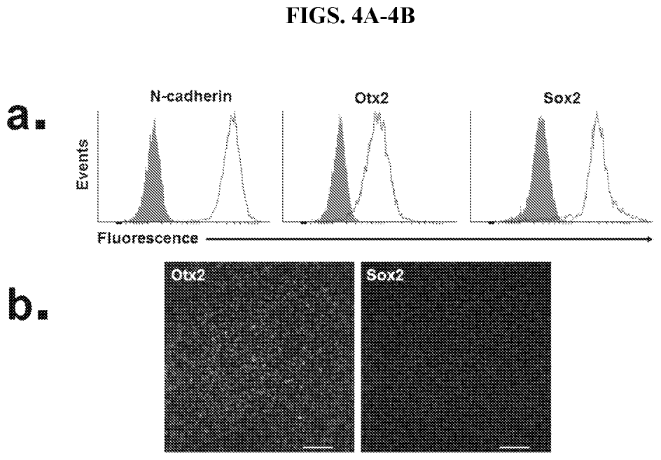

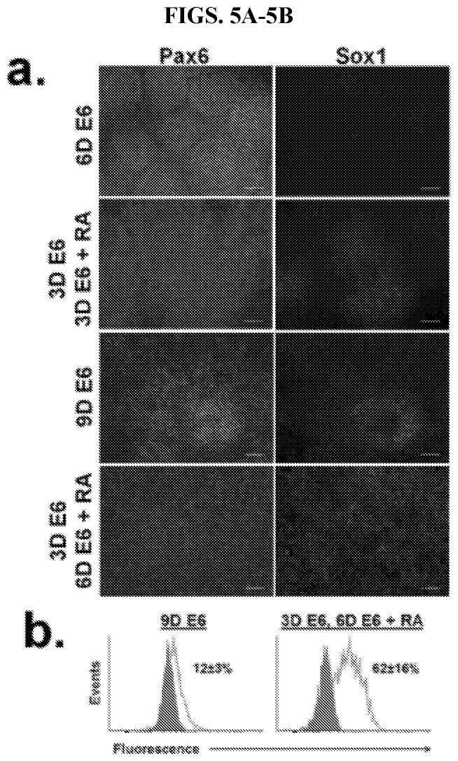

Differentiation to neuroepithelium can be conducted under adherent conditions or using embryoid body (EB)-based methods (Pankratz et al (2007), Stem Cells, 25: 1511-1520. EB-based methods can take up to 17 days to yield definitive neuroepithelium, and controlling EB differentiation in a reproducible, scalable fashion is a challenging prospect (see, e.g., Bratt-Leal et al (2009), Biotechnology Progress 25: 43-51. Recent reports in adherent differentiation have demonstrated more rapid neuralization using small molecules and recombinant proteins, yielding >80% neuroepithelium after 11 days (Chambers et al (2009), Nature Biotechnology, 27:275-280. The original protocols used SB431542 (an inhibitor of TGFβ signaling) and noggin (an inhibitor of bone morphogenetic protein (BMP) signaling), and follow-up protocols (e.g., Kim et al (2010), Stem Cell Reviews 6:270-281) have replaced noggin with the small molecules dorsomorphin or LDN-193189. Such factors were shown in these previous studies to inhibit endoderm and mesoderm formation and therefore promote high neuroectoderm efficiency. However, we reasoned that hPSCs maintained in defined medium under feeder-independent conditions might be naturally biased towards forming neuroectoderm and not necessarily require these exogenous factors. Therefore, we cultured H9 hESCs in E8 medium (Chen et al, supra), verified their expression of pluripotency markers (FIGS. 1a and 1b) and subcultured these cells onto Matrigel®-coated plates at a high density (2×10⁵ cells/cm²) in E8 medium containing ROCK inhibitor (FIG. 2a). To initiate differentiation the following day, E8 medium was completely replaced with a formulation in which the factors in E8 medium which maintain pluripotency (FGF2 and TGFβ1) were removed—this resultant medium is denoted as “E6” (see Materials and Methods for details). To determine the necessity of SMAD inhibitors for neuroectoderm formation under these conditions, we initially tested neuroectoderm specification using E6 medium, E6 containing SB431542, or E6 containing SB431542 and noggin and monitored differentiation by RT-PCR, immunocytochemistry, and flow cytometry. RT-PCR revealed expression of pluripotency genes POU5F1 (OCT4) and NANOG in undifferentiated hESCs, and expression of these genes gradually decreased for all differentiation conditions (FIG. 2b). Expression of NANOG was also shown to decrease throughout differentiation (FIG. 3). T (encoding BRACHYURY), which is expressed in primitive streak mesoderm, was strongly detected in E6 medium cultures at day 2 and reduced T expression was observed at this time point in the presence of SB431542 and noggin. As differentiation progressed, expression of this mesodermal marker decreased and ultimately disappeared under all conditions (FIG. 2b). In contrast, mRNA for PAX6, a neuroectoderm fate determinant (Zhang et al (2010), Cell Stem Cell, 7:90-100) was first detected, by RT-PCR, after 2 days of differentiation (FIG. 2b), and became strongly expressed by day 4 of differentiation for all conditions. SOX2, which is expressed in both undifferentiated hPSCs and neuroectoderm, was detected at all time points, while S^{OX1} (a marker of neuroectoderm), OTX2 (a marker of midbrain and forebrain), and FOXG1 (a marker of forebrain) were also expressed in undifferentiated cells and throughout differentiation. However, while SOX2 protein was abundantly detected in hESCs by immunocytochemistry (FIG. 1a), PAX6, SOX1, and OTX2 proteins were not expressed in the pluripotent state (data not shown). Rostral character was further confirmed for all differentiation conditions by the absence of HOXB4 transcript, a hindbrain marker (FIG. 2b). The ventral transcription factor OLIG2 was not detected by RT-PCR, indicating a lack of dorsal/ventral lineage patterning, and the definitive endoderm marker SOX/7 was not detected. To quantify neural differentiation, we analyzed PAX6 expression by flow cytometry (FIG. 2c). PAX6 expression was not detected in the H9 hESCs at day 2 of differentiation under any differentiation conditions (E6, E6+SB432542, or E6+SB431542+noggin). By day 3 of differentiation in E6 medium alone, 72±4% of cells expressed PAX6. At days 4 and 6 of differentiation, PAX6 expression was >90% under all differentiation conditions. Notably, differentiation of H9 hESCs in E6 medium alone yielded nearly uniform expression of PAX6 after 6 days of differentiation (98±2%). IMR90-4 iPSCs maintained in E8 medium could also be differentiated to neuroepithelium with high efficiency using E6 medium alone (87±9%; FIG. 2c). Cells at this stage were also 100±0% N-Cadherin+, 95±0% OTX2⁺, and 98±1% SOX2⁺ (FIG. 4). Immunocytochemistry confirmed nuclear PAX6 expression and widespread neural rosette formation as indicated by polarization of N-Cadherin towards an inner lumen (FIG. 2d), which is a hallmark of in vitro neuroepithelium formation (Koch et al (2009), Proc. Natl Acad. Sci USA, 106:3225-3230). Interestingly, although SOX1 transcript was detectable throughout the differentiation process, Sox1 protein was still not expressed by day 6 (FIG. 5a). Its expression became localized to small regions by day 9, although only 12±3% of the total cells exhibited detectable expression by flow cytometry (FIG. 5b). If retinoic acid was added to facilitate differentiation, increased SOX1 expression was observed (up to 62±16% by day 9; FIGS. 5a and 5b). Previous reports have demonstrated that PAX6 expression precedes SOX1 expression in vitro (Pankratz et al (2007), Stem Cells, 25, 1511-1520) and in vivo (Zhang et al. (2010), Cell Stem Cell, 7:90-100. Therefore, the E6 differentiation procedure is in good agreement with developmental timing principles and previously established differentiation protocols.

### Example 3 Directed Neural Differentiation Under Completely Defined, Xeno-Free Conditions

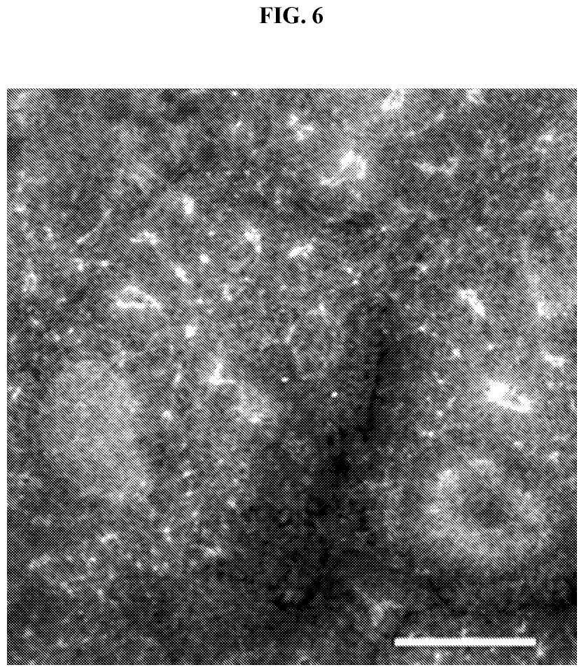

All experiments described above in Example 2 utilized Matrigel® as the culture substrate during maintenance and differentiation. Thus, to construct a completely defined system, we maintained H1 and H9 hESCs in E8 medium on recombinant vitronectin peptide (VTN-NC) and then differentiated the cells in E6 medium as described above but replaced Matrigel® with VTN-NC. Differentiation on this defined surface yielded 90±1% PAX6⁺ cells from H1 hESCs and 99±1% PAX6⁺ cells from H9 hESCs after 6 days (FIG. 2c), and neural rosette formation was also observed under these conditions (FIG. 2d and FIG. 6). Thus, the effectiveness of the differentiation procedure does not depend on the use of Matrigel® as a substrate. Overall, this procedure yields highly pure definitive neuroepithelium under completely defined and scalable adherent conditions.

### Example 4 Pluripotent Stem Cell Culture Conditions Affect the Efficiency of Neural Differentiation

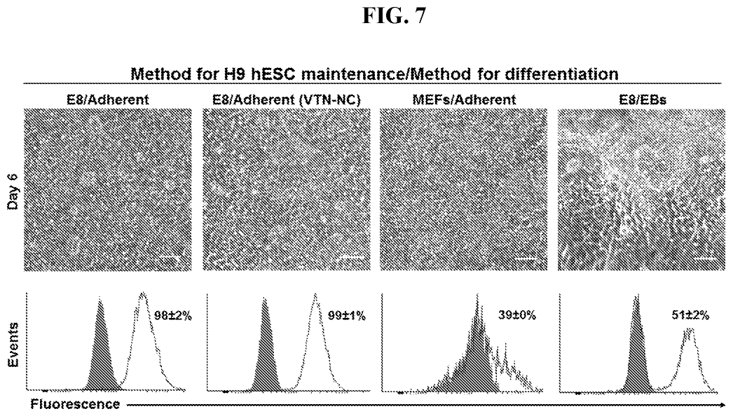

We sought to determine if the efficiency of differentiation protocol was influenced by the pluripotent stem cell culturing conditions we used. To assess the impact of pluripotent stem cell culture conditions on our differentiation protocol, we tested the E6 differentiation protocol described above on H9 hESCs that had been maintained in the undifferentiated state on mouse embryonic fibroblasts (MEFs). After differentiation in E6 medium for 4 days, no PAX6⁺ cells were detected by flow cytometry (data not shown). After 6 days of differentiation, some regions of cells possessed putative neuroepithelial morphology but not polarized rosette formation and only 39±0% of cells were PAX6+(FIG. 7). Thus, hESCs maintained on MEFs do not efficiently form neuroepithelium when differentiated in E6 medium alone. We also assessed the effect of adherent versus EB conditions on neural differentiation by forming EBs from H9 hESCs maintained in E8 medium, differentiating these EBs for 4 days in E6 medium as free-floating aggregates, and plating the EBs onto Matrigel®-coated dishes for an additional 2 days of differentiation. Some EBs produced regions with polarized rosette morphology but only 51±2% of the total cell population was PAX6⁺ (FIG. 7). Our data, therefore, suggest that maintenance under feeder-independent conditions enhances the efficiency of our protocol for promoting neuroepithelial differentiation. EB-based methods can also yield neuroepithelium and polarized rosette structures using E6 medium but appeared to be less efficient than previous reports in which hESCs were cultured under feeder-dependent conditions (Pankratz et al, supra).

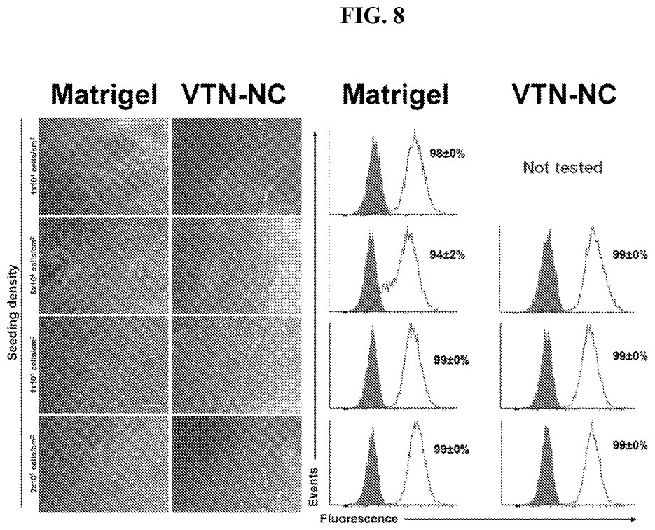

Finally, we sought to determine if seeding density was an important variable for efficient neuroepithelial differentiation under adherent conditions. H9 hESCs seeding density could be reduced to 1×10⁵ cells/cm² on either Matrigel® or VTN-NC and cells were still competent to form neuroepithelium with >98% Pax6⁺ expression (FIG. 8). Seeding densities of 1×10⁴ or 5×10⁴ cells/cm² led to decreased cell outgrowth and a qualitative decrease in the amount of rosette formation (FIG. 8). Thus, a high initial seeding density is important for achieving definitive neuroepithelium, similar to previous reports (e.g., Chambers et al, supra).

### Example 5 Transferrin, but not Insulin, can be Omitted for Neural Differentiation

We sought to determine if either insulin or transferrin were strictly necessary in the neural differentiation medium and method we developed. Accordingly, we compared our neural differentiation protocol in E6 medium and two different E6 minus one component media formulations, which had the same composition as the E6 medium, but which omitted either insulin or transferrin.

H9 hESCs were seeded at 2×10⁵ cells/cm² on Matrigel® in E8 medium plus ROCK inhibitor as described above. The following day, the medium was changed to E6 medium, E6 medium minus transferrin, or E6 medium minus insulin, and media were replaced every day through day 6. On day 6, the cells were fixed and analyzed by flow cytometry for the expression of the markers PAX6, OTX2, N-Cadherin, and SOX2. Also, the morphological characteristics of the cells at day 6 were assessed by bright field microscopy.

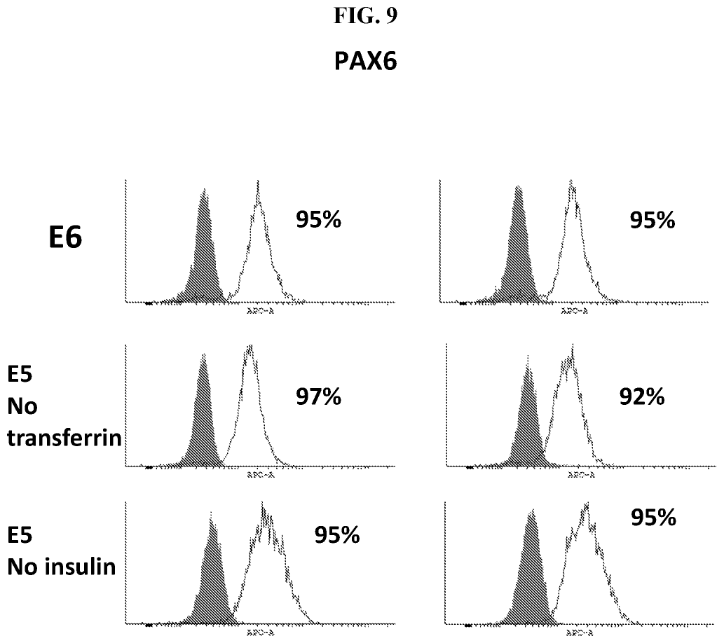

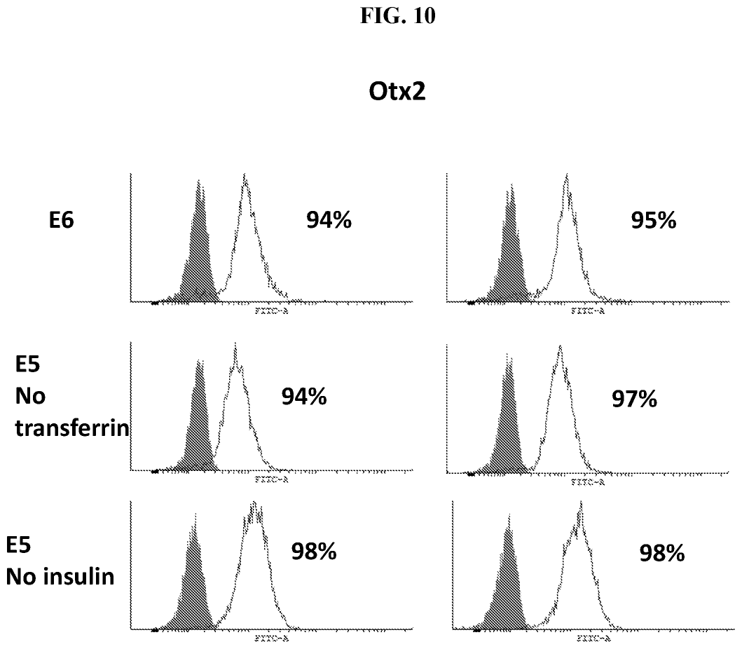

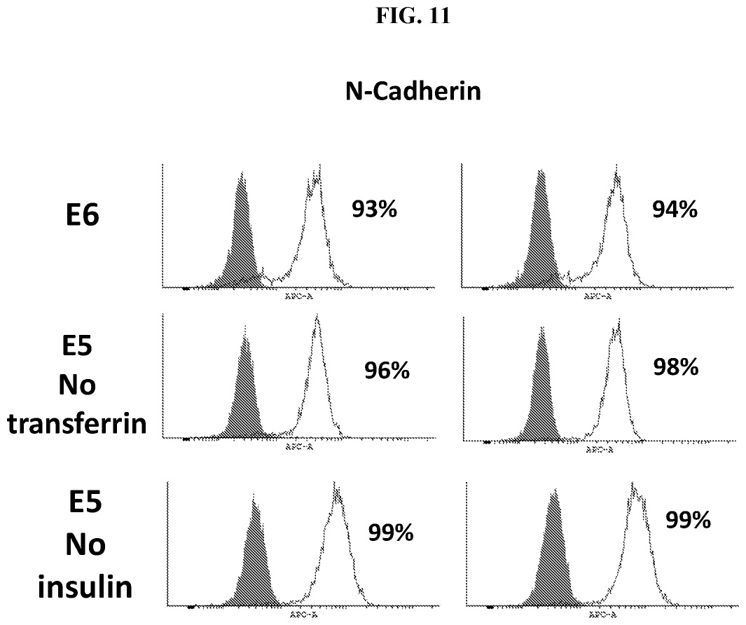

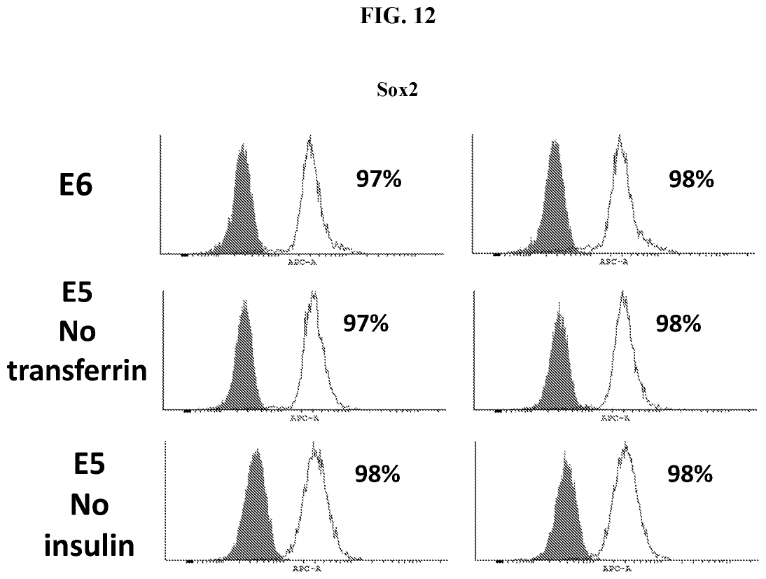

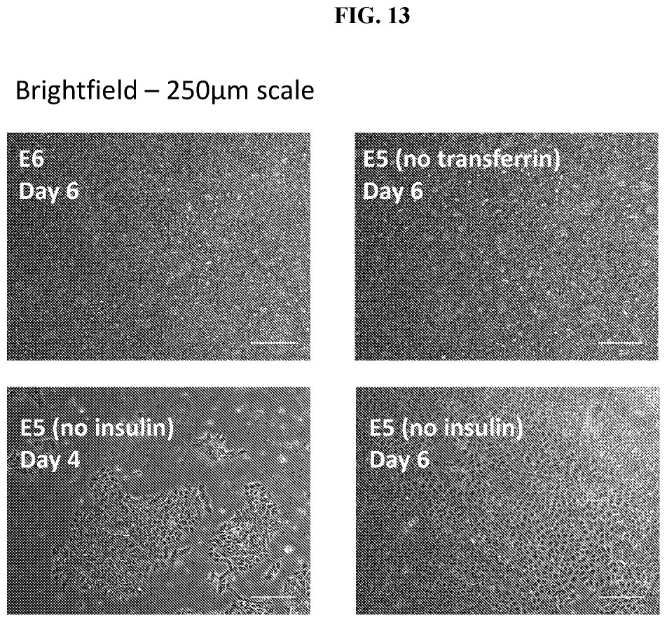

As shown in FIGS. 9-12, cells under all three media conditions expressed similar, high levels of PAX6, OTX2, N-Cadherin, and SOX2. However, in the absence of insulin, there was a very sharp decrease in the level of cell viability as shown in FIG. 13. Further, polarized rosettes were observed only in cells cultured under E6 or E6 minus transferrin conditions.

Thus, it was concluded that the presence of insulin is critical for cell viability, despite the fact that a small subset of cells surviving in the absence of insulin do express the expected markers. On the other hand, it appears that transferrin is fully dispensable for differentiation of hPSCs into neural stem cells in the method we have developed.

### Example 6 Ascorbate, but not Selenium can be Omitted for Neural Differentiation

In a further effort to define the minimal culture medium supplements needed for efficient neural differentiation of human pluripotent stem cells, we examined whether differentiation could be carried out in either of two “E4” formulations: (1) Bicarbonate-buffered DMEM/F12+Insulin+Selenium and (2) Bicarbonate-buffered DMEM/F12+Insulin+Ascorbic Acid.

H9 hESCs were seeded at a density of 100,000 cells/cm² on VTN-NC in E8 medium plus ROCK inhibitor. The following day, cells were changed to either of the above-mentioned E4 media, where the concentrations of selenium or ascorbic acid were at the standard concentrations used in E6 medium. The medium was changed every day until day 6 when flow cytometry was used to assess expression of neural differentiation markers under different media conditions.

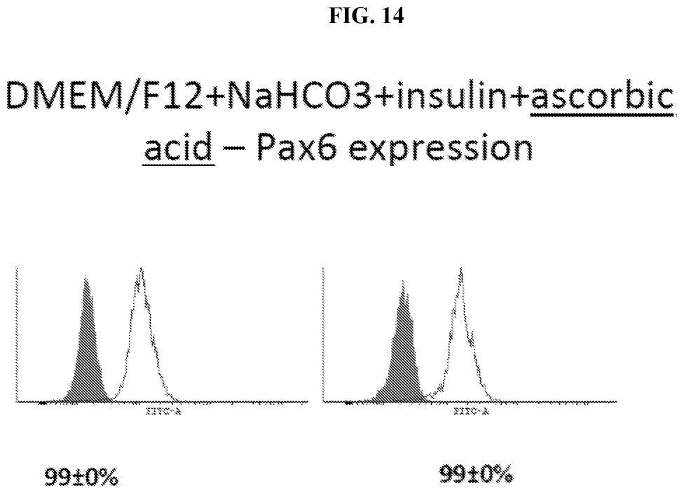

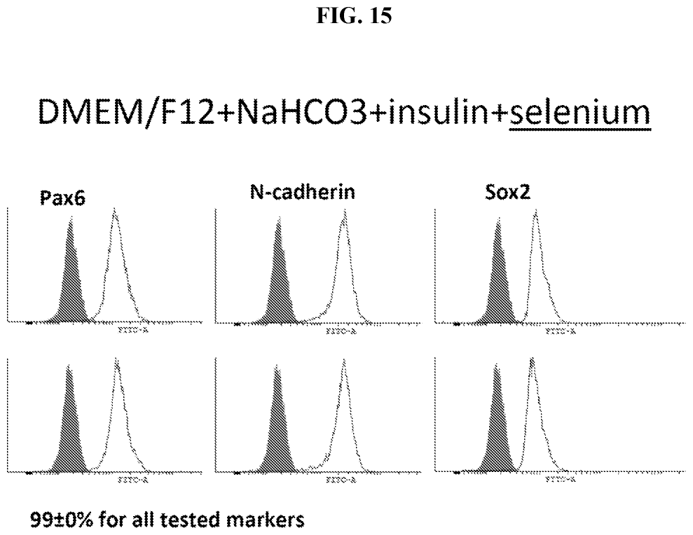

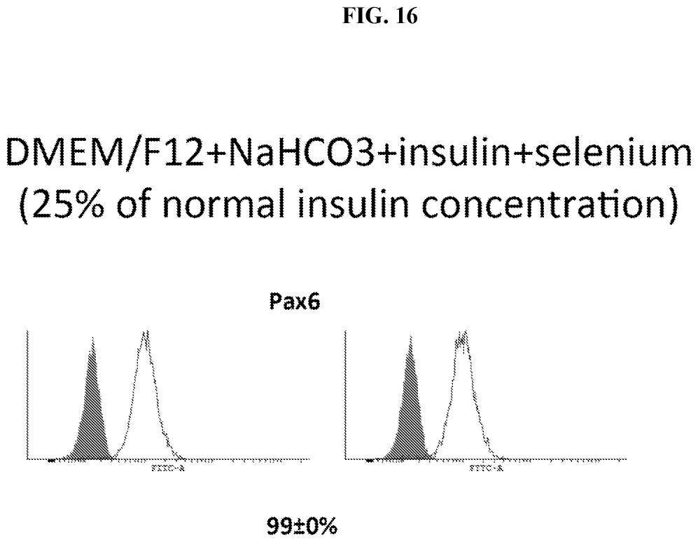

As shown in FIG. 14, expression of PAX6 in the absence of selenium was equivalent to that observed in cells differentiated in full E6 medium. Likewise, as shown in FIG. 15, 99% of the cell population obtained by culture in an E4 formulation medium lacking ascorbate but containing selenium expressed the neuroepithelial markers PAX6, N-cadherin, and SOX2. Interestingly, we also found that in an E4 formulation containing selenium, the concentration of insulin could be reduced to 25% of the level used in E6 medium without any apparent effect on the level of PAX6 expression in the differentiated cell population (FIG. 16).

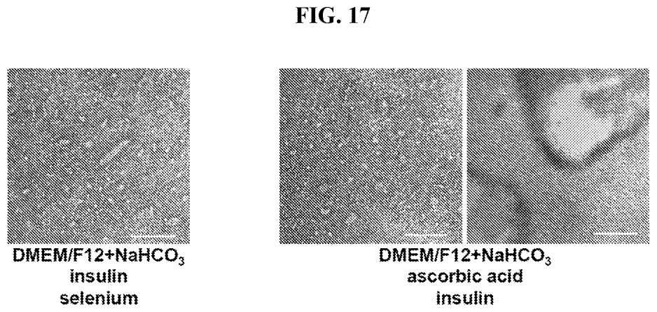

We also observed that despite the fact that E4 medium containing ascorbate but lacking selenium yielded neural rosettes and high levels of PAX expression, a marked loss of viability was observed relative to cells cultured in E4 lacking ascorbate but containing selenium (FIG. 17). Based on these results, we concluded that an E4 formulation can omit ascorbate but not selenium. Moreover, the concentration of insulin necessary to support differentiation and viability can be reduced at least four fold relative to the insulin concentration used in the full E6 medium formulation.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

1. A method for directed differentiation of human pluripotent stem cells into neural stem cells, comprising:
(a) seeding human pluripotent stem cells (hPSCs) onto a xenogen-free substrate and culturing the cells in the presence of a Rho kinase inhibitor for a first culture period of about one day;
(b) culturing the cells of step (a) for a second culture period of about 4 to about 6 days on the xenogen-free substrate in a neural differentiation medium to obtain a culture of neural stem cells wherein at least 90% of the cells are Pax6+.

2. The method of claim 1, wherein the xenogen-free substrate comprises a vitronectin, a vitronectin fragment, a vitronectin peptide, a recombinant vitronectin peptide, or a synthetic peptide.

3. The method of claim 2, wherein the xenogen-free substrate comprises a recombinant vitronectin peptide.

4. The method of claim 1, wherein the hPSCs are seeded at a density of at least about 1×10⁵ cells/cm².

5. The method of claim 1, wherein the hPSCs in step (a) are cultured in the presence of ascorbate, transferrin, a fibroblast growth factor, and TGFβ1.

6. The method of claim 5, wherein the fibroblast growth factor is FGF2.

7. The method of claim 5, wherein the cells in step (b) are cultured in the absence of the fibroblast growth factor and TGFβ1.

8. The method of claim 5, wherein the cells in step (b) are cultured in the absence of the transferrin, fibroblast growth factor, and TGFβ1.

9. The method of claim 1, wherein the cells of step (b) form neural rosettes after about 4 to about 6 days of culture on the xenogen-free substrate in the neural differentiation medium.

10. A method for directed differentiation of human pluripotent stem cells into neural stem cells, comprising:
(a) seeding human pluripotent stem cells (hPSCs) onto a xenogen-free substrate comprising a recombinant vitronectin peptide and culturing the cells in the presence of a Rho kinase inhibitor for a first culture period of about one day;
(b) culturing the cells of step (a) for a second culture period of about 4 to about 6 days on the xenogen-free substrate in a neural differentiation medium a culture of neural stem cells wherein at least 90% of the cells are Pax6+.

11. The method of claim 10, wherein the hPSCs are seeded at a density of at least about 1×10⁵ cells/cm².

12. The method of claim 10, wherein the hPSCs in step (a) are cultured in the presence of ascorbate, transferrin, a fibroblast growth factor, and TGFβ1.

13. The method of claim 12, wherein the fibroblast growth factor is FGF2.

14. The method of claim 12, wherein the cells in step (b) are cultured in the absence of the fibroblast growth factor and TGFβ1.

15. The method of claim 12, wherein the cells in step (b) are cultured in the absence of the transferrin, fibroblast growth factor, and TGFβ1.

16. The method of claim 10, wherein the cells of step (b) form neural rosettes after about 4 to about 6 days of culture on the xenogen-free substrate in the neural differentiation medium.

17. A method for directed differentiation of human pluripotent stem cells into neural stem cells, comprising:
(a) seeding human pluripotent stem cells (hPSCs) at a density of about 2×10⁵ cells/cm² onto a xenogen-free substrate comprising a recombinant vitronectin peptide and culturing the cells in a neural differentiation medium comprising a Rho kinase inhibitor, ascorbate, transferrin, a fibroblast growth factor, and TGFβ1 for a first culture period of about one day;
(b) culturing the cells of step (a) for a second culture period of about 4 to about 6 days on the xenogen-free substrate in the neural differentiation medium of step (a) without the fibroblast growth factor and TGFβ1, and optionally without transferrin, to obtain a culture of neural stem cells wherein at least 90% of the cells are Pax6+.

18. The method of claim 1, wherein the fibroblast growth factor is FGF2.

19. The method of claim 17, wherein the neural differentiation medium of step (b) is without transferrin.

20. The method of claim 1, wherein the cells of step (b) form neural rosettes after about 4 to about 6 days of culture on the xenogen-free substrate in the neural differentiation medium.
